(19) United States
(12) Patent Application Publication
(10) Pub. No.: US 2015/0208954 A1
(43) Pub. Date: Jul. 30, 2015

(54) APPARATUS FOR ANALYZING HUMAN MOTION

(71) Applicant: ELECTRONICS AND TELECOMMUNICATIONS RESEARCH INSTITUTE, Daejeon (KR)

(72) Inventors: Myung-Gyu KIM, Daejeon (KR); Ye-Jin KIM, Daejeon (KR); Seong-Min BAEK, Daejeon (KR); Jong-Sung KIM, Daejeon (KR); Il-Kwon JEONG, Daejeon (KR)

(21) Appl. No.: 14/596,369

(22) Filed: Jan. 14, 2015

(30) Foreign Application Priority Data
Jan. 27, 2014 (KR) 10-2014-0009898

(51) Int. Cl. A61B 5/11; A61B 5/024; A61B 5/00; A61B 5/0488; A61B 5/06; A61B 5/021; A61B 5/0402

(52) U.S. Cl. CPC A61B 5/11; A61B 5/021; A61B 5/024; A61B 5/0402; A61B 5/0488; A61B 5/06; A61B 5/0022; A61B 5/7278

(57) ABSTRACT

The present invention provides an apparatus for analyzing human motion including a bio-sensor configured to transmit a bio-signal by being attached to a body part; a receiver configured to receive the bio-signal; and a controller configured to determine position and velocity of the bio-sensor based on the bio-signal and estimate position and velocity of the joint adjacent to the body part based on the position and velocity of the bio-sensor. Accordingly, the present invention can reduce restrictions in movements by using a wireless bio-signal without having any problem in being hidden.

### CROSS REFERENCE TO RELATED APPLICATION

This application claims the benefit of Korean Patent Application No. 10-2014-0009898, filed on Jan. 27, 2014, entitled “Apparatus for analyzing human motion”, which is hereby incorporated by reference in its entirety into this application.

### BACKGROUND

1. Technical Field

The present invention relates to a technology for analyzing human motion and more particularly, to an apparatus for analyzing human motion which estimates position and velocity of a joint based on bio-signals of bio-sensors which are attached to a body.

2. Description of the Related Art

A technology for analyzing human motion is generally needed to obtain 3-D positions of joints used for human performances of dance, sports, acting and the like. Human motion is defined by a time function of 3-D position and 3-D velocity of a joint. Here, the obtained 3-D time function of the joint can be applied to games through virtual characters and E-learnings through comparison with actual facts.

Conventional human motion analysis is divided into a marker-free motion analysis in which markers are not attached to body parts and a marker-based motion analysis in which marker are attached to body parts. The marker-free motion analysis does not impose many restrictions on user's movements but causes many position errors, while the marker-based motion analysis causes less position errors but requires many restrictions on user's movements. Particularly, since the marker-based motion analysis needs to attach numerous markers to joints and causes restrictions on movements due to those attached markers, some markers cannot be in the line of sight with particular postures or can be hidden by lamps or lights, thereby causing errors in positioning joints.

The conventional marker-based motion analysis system is disclosed in US Patent Publication No. 2007-0058839 (System and method for capturing facial and human motion).

On the other hand, a bio-sensor can be embedded inside the skin to obtain bio-signals of a user and the bio-signals can be used to analyze human motion. Accordingly, an apparatus for analyzing human motion having functions of the conventional marker-based human motion analysis and utilizing wireless bio-signal communications is demanded.

### SUMMARY

Embodiments of the present invention allow to provide an apparatus for analyzing human motion which can resolve the problems of restrictions of movements and hidden markers for particular postures or lights, associated with marker-based human motion analysis.

Embodiments of the present invention allow to provide an apparatus for analyzing human motion having extended functions by utilizing a conventional bio-sensor.

In an aspect of the present invention, the present invention provides an apparatus for analyzing human motion comprising: a bio-sensor configured to transmit a bio-signal by being attached to a body part; a receiver configured to receive the bio-signal; and a controller configured to determine position and velocity of the bio-sensor based on the bio-signal and estimate position and velocity of the joint adjacent to the body part based on the position and the velocity of the bio-sensor.

In an embodiment of the present invention, the controller identifies the bio-sensor transmitting the bio-signal by analyzing a time series pattern of the bio-signal.

In an embodiment of the present invention, the controller determines position of the bio-sensor by a triangulation.

In an embodiment of the present invention, the controller calculates position of the bio-sensor by using Equation of Ps(t)=(Xs(t), Ys(t), Zₛ(t)), in which Ps(t)=(Xs(t), Ys(t), Zₛ(t)) is the position of the bin-sensor at time t.

In an embodiment of the present invention, the controller determines velocity of the bio-sensor by the frequency shift-based Doppler effect.

In an embodiment of the present invention, the controller calculates velocity of the bio-sensor by using Equation of

$${V}_{s}^{1} = ( \frac{{X}_{s} ( t + δ t ) - {X}_{s} ( t )}{δ t} , \frac{{Y}_{s} ( t + δ t ) - {Y}_{s} ( t )}{δ t} , \frac{{Z}_{s} ( t + δ t ) - {Z}_{s} ( t )}{δ t} ) ,$$

in which Vₛ¹ is velocity vector of the bio-sensor at time t.

In an embodiment of the present invention, the controller calculates velocity of the bio-sensor by using Equation of

$${V}_{s}^{2} = {\sum }_{i = 1}^{3} \frac{Δ {f}_{i}}{{f}_{i}} \frac{{Q}_{i} - {P}_{s}}{‖ {Q}_{i} - {P}_{s} ‖}$$

$$‖ {Q}_{i} - {P}_{s} ‖ = {\sum }_{i = 1}^{3} \sqrt{{( {X}_{i} - {X}_{s} )}^{2} + {( {Y}_{i} - {Y}_{s} )}^{2} + {( {Z}_{i} - {Z}_{s} )}^{2}} ,$$

in which Vₛ² is velocity vector of the bio-sensor at time t, fᵢ is frequency of the bio-signal, Qᵢ-Pₛ is displacement vector of the bio-sensor which is a linear independent.

In an embodiment of the present invention, the controller interpolates velocity of the bio-sensor by using the Equation of Vₛ=aVₛ¹(1−a)Vₛ² (0≦a≦1), in which Vₛ is velocity vector of the bio-sensor and a is a predetermined value by a user.

In an embodiment of the present invention, wherein the controller estimates velocity of the joint based on the position of the joint.

In an embodiment of the present invention, the controller estimates position of the joint based on the position of the bio-sensor included in frames measured continuously in time intervals.

In another aspect of the present invention, the present invention provides a method for analyzing human motion comprising: receiving a bio-signal from a bio-sensor attached to a body part; determining position of the bio-sensor based on the bio-signal; determining velocity of the bio-sensor based on the bio-signal; and estimating position and velocity of the joint based on the position and the velocity of the bio-sensor.

In an embodiment of the present invention, the method for analyzing human motion further comprises identifying the bio-sensor transmitting the bio-signal by analyzing a time series pattern of the bio-signal.

An embodiment of the present invention can reduce restrictions in movements by using wireless bio-signals without having any problem in being hidden.

An embodiment of the present invention can also reduce errors caused from being hidden by using wireless bio-signals without having any problem in being hidden.

An embodiment of the present invention can not only analyze human motion by using bio-signals but also utilize original physical activity information included in the bio-signals.

### BRIEF DESCRIPTION OF THE DRAWINGS

FIG. 1 illustrates configuration of an apparatus for analyzing human motion according to an embodiment of the present invention.

FIG. 2 illustrates an apparatus for analyzing human motion according to an embodiment of the present invention.

FIG. 3 illustrates bio-signal time series patterns according to an embodiment of the present invention.

FIG. 4 illustrates an example of inducing position and velocity of a joint according to another embodiment of the present invention.

FIG. 5 is a flowchart illustrating a method for analyzing human motion according to an embodiment of the present invention,

### DESCRIPTION OF THE EXEMPLARY EMBODIMENTS

The above and other objects, features and advantages of the present invention will become more apparent to those of ordinary skill in the art by describing in detail exemplary embodiments thereof with reference to the accompanying drawings. While the present invention has been described with reference to particular embodiments, it is to be appreciated that various changes and modifications may be made by those skilled in the art without departing from the spirit and scope of the present invention, as defined by the appended claims and their equivalents. The terms used in the description are intended to describe certain embodiments only, and shall by no means restrict the present invention. Throughout the description of the present invention, when describing a certain technology is determined to evade the point of the present invention, the pertinent detailed description will be omitted. Throughout the description of the present invention, components are rendered the same reference number that are the same or are in correspondence, regardless of the figure number in order to facilitate a throughout understanding.

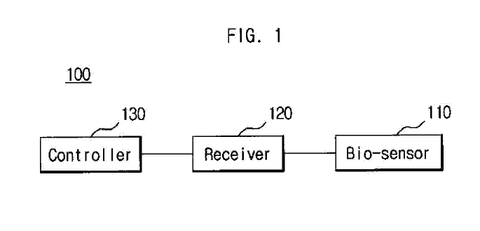

FIG. 1 illustrates configuration of an apparatus for analyzing human motion according to an embodiment of the present invention.

Referring to FIG. 1, an apparatus for analyzing human motion 100 comprises a bio-sensor 110, a receiver 120 and a controller 130.

The bio-sensor 110 collects information to determine identification or state of individuals. The bio-sensor 110 may recognize fingerprint, iris and the like or determine blood pressure, electromyogram(EMG), heart rate, blood sugar, electrocardiogram(ECG) and the like.

The bio-sensor 110 detects a bio-signal from a body part by being attached to a body part and transmits the bio-signal to the receiver 120. The bio-sensor 110 may be attached to a plurality of body parts. The plurality of bio-sensors 110 transmit bio-signals from each body part to the receiver 120. The bio-sensor 110 may wirelessly transmit the bio-signal periodically or aperiodically.

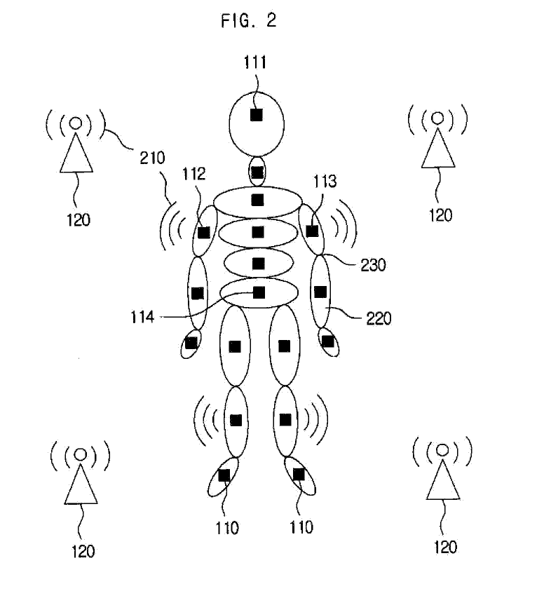

The body part means each part configuring a human body. Since the body part is necessary to recognize human motion, the body part of the present invention may be a part of human body which can move independently. For example, the body part which can move independently means the body part which can move by its own even though other body parts do not move and performs the same movement when it moves. As shown in FIG. 2, face, neck, thighs, legs, arms, hands and the like can be body parts where the bio-sensor 110 can be attached. A body part where the bio-sensor 110 is attached can be divided or combined depending on amount of data to obtain bio-signals.

The bio-signal includes information of a human body. The bio-signal includes information of a human body which can be obtained from the bio-sensor 110. The bio-signal can include static information representing characteristics of a human such as fingerprint, iris and the like or dynamic information representing state changes of a human such as blood pressure, electromyogram(EMG), heart rate, blood sugar, electrocardiogram(ECG) and the like.

The receiver 120 receives a bio-signal detected from a body part from the bio-sensor 110. When a plurality of bio-sensors 110 are attached to a plurality of body parts, the receiver 120 receives a plurality of bio-signals from the plurality of bio-sensors 110. The receiver 120 transmits the received bio-signals to the controller 130.

FIG. 2 illustrates an apparatus for analyzing human motion according to an embodiment of the present invention.

Referring to FIG. 2, the bio-sensors 110 are attached to each body part and wirelessly transmit/receive bio-signals to/from the receiver 120. Since the bio-sensors 110 communicate wirelessly with the receiver 120, they consistently transmit bio-signals, which are generated whenever the human body moves, and the controller 130 identifies human motion based on the received bio-signals.

Communication between the bio-sensor 110 and the receiver 120 can be made through wired communication or wireless communication. In case of wireless communication, the communication can be made through Bluetooth, ultra-low power(ULP), Wi-Fi, ZigBee, RF, infrared communication(IrDA) and the like. In addition, various methods for transmitting and receiving information with external devices can be included as the wireless communication.

The controller 130 determines position and velocity of the bio-sensor 110 based on the bio-signal and estimates position and velocity of the joint adjacent to the body part based on the position and the velocity of the bio-sensor 110. Human motion can be defined by a time function of 3-D position and 3-D velocity of a joint when the human being performs any movement. Thus, position and velocity of the joint adjacent to a body part estimated by the controller 130 is based on the human motion analysis.

The controller 130 identifies origins of the received plurality of wireless bio-signals. The controller 130 identifies which bio-sensor 110 the bio-signals, received from a plurality of body parts, are from. The controller 130 thus identifies each bio-sensor 110 by analyzing a time series pattern for amplitude of the bio-signal.

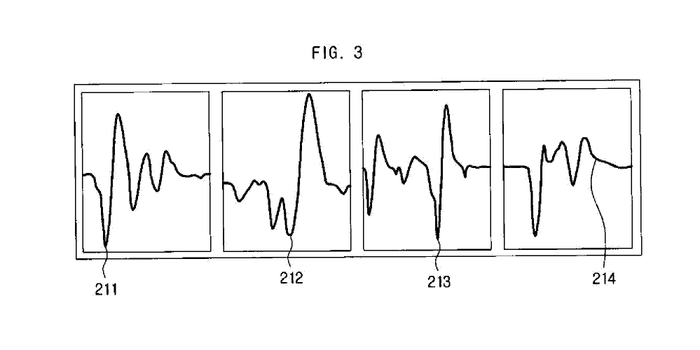

FIG. 3 illustrates bio-signal time series patterns according to an embodiment of the present invention.

Referring to FIG. 3, time series patterns are illustrated for 4 bio-signal amplitudes, which are the first bio-signal 211 transmitted by the first bio-sensor 111 of face, the second bio-signal 212 transmitted by the second bio-sensor 112 of left arm, the third bio-signal 213 transmitted by the third bio-sensor 113 of right arm, and the fourth bio-signal 214 transmitted by the fourth bio-sensor 114 of abdomen. The first to the fourth bio-sensors(111 to 114) detect bio-signals from the corresponding body parts, respectively and each of the first to the fourth bio-signals(211 to 214) represents a different'time series amplitude pattern. The controller 130 determines which bio-sensor 110 each received bio-signal is from by matching the received bio-signals on the conventional time series patterns to identify the origin of each of the plurality of bio-signals.

The controller 130 determines positions of the bio-sensors 110 which transmitted the plurality of wireless bio-signals. The controller 130 determines positions of the bio-sensors 110 in the 3-dimension. The controller 130 determines 3-D positions of the bio-sensors 110 by using a triangulation.

3-D positions of n number of receivers 120 are assumed to be represented by Pᵢ=(Xᵢ, Yᵢ, Zᵢ) (i=1, . . . , n). A signal from the bio-sensor 110, S, includes transmitted time information. The controller 130 calculates time difference by using time information received from the receiver 120 and calculates distance from the receiver 120 to the bio-sensor 110 by multiplying time difference by light velocity. The controller 130 calculates 3-D position of the bio-sensor 110 by using the distance from the receiver 120 to the bio-sensor 110 and the triangulation. When the 3-D position of the bio-sensor 110, S, is represented by Pₛ=(Xₛ, Yₛ, Zₛ), it is calculated by the following Equation at time t:

Pₛ(t)=(Xₛ(t), Yₛ(t), Zₛ(t)) (1)

in which Pₛ(t)=(Xₛ(t), Yₛ(t), Zₛ(t)) is position of the bio-sensor at time t.

The controller 130 determines velocity of the bio-sensor 110 transmitting a plurality of wireless bio-signals. The controller 130 determines velocity of the bio-sensor 110 in the 3-dimension. The controller 130 determines 3-D position of the bio-sensor 110 by using frequency shift-based Doppler effect. The controller 130 calculates velocity in two ways and interpolates the calculated result in order to obtain accurate velocity of the bio-sensor 110.

The controller 130 calculates the velocity of the first bio-sensor 110 as 3-D velocity vector of the bio-sensor 110, S, at time t. The controller 130 calculates 3-D velocity vector through the following Equation:

$${V}_{s}^{1} = ( \frac{{X}_{s} ( t + δ t ) - {X}_{s} ( t )}{δ t} , \frac{{Y}_{s} ( t + δ t ) - {Y}_{s} ( t )}{δ t} , \frac{{Z}_{s} ( t + δ t ) - {Z}_{s} ( t )}{δ t} ) \qquad ( 2 )$$

in which, Vₛ¹ means velocity vector of the bio-sensor at time.

The controller 130 calculates velocity of the second bio-sensor 110 as 3-D velocity vector of the bio-sensor 110 by using Doppler shifts. It is assumed that frequency of the bio-signal, transmitted from the stalled bio-sensor 110 S and received from the receiver 120 I, is f, and frequency of the bio-sensor, transmitted from moving bio-sensor 110 S and received from the receiver 120 I, is (fᵢ+fᵢ). When the bio-sensor 110 S is stalled, fᵢ is 0; when it moves to be closer to the receiver 1201, fᵢ is a positive number; and when it moves to be away from the receiver 120 I, fᵢ is a negative number. The controller 130 selects 3 linear independent displacement vectors(Qᵢ−Pₛ) from n number of displacement vectors (Pᵢ−Pₛ). The controller 130 calculates 3-D velocity vector of the bio-sensor 110 S by the following Equation:

$${V}_{s}^{2} = {\sum }_{i = 1}^{3} \frac{Δ {f}_{i}}{{f}_{i}} \frac{{Q}_{i} - {P}_{s}}{‖ {Q}_{i} - {P}_{s} ‖} \\ ‖ {Q}_{i} - {P}_{s} ‖ = {\sum }_{i = 1}^{3} \sqrt{{( {X}_{i} - {X}_{s} )}^{2} + {( {Y}_{i} - {Y}_{s} )}^{2} + {( {Z}_{i} - {Z}_{s} )}^{2}} \qquad ( 3 )$$

in which, Vₛ² velocity vector of the bio-sensor at time t, fᵢ means frequency of the bio-sensor, and Qᵢ−Pₛ means linear independent displacement vector of the bio-sensor.

The controller 130 interpolates the 3-D velocity vector Vₛ obtained by using those two, methods. The controller 130 improves accuracy of the velocity vector Vₛ by using the following Equation in which a is selected by a user from 0 to 1:

Vₛ=aVₛ¹+(1−a)Vₛ² (0≦a≦1) (4)

in which, Vₛ means velocity vector of the bio-sensor and a means a predetermined value selected by a user.

The controller 130 calculates 3-D position and 3-D velocity of a joint by using the 3-D position and the 3-D velocity of the corresponding bio-sensor 110 attached to a body part.

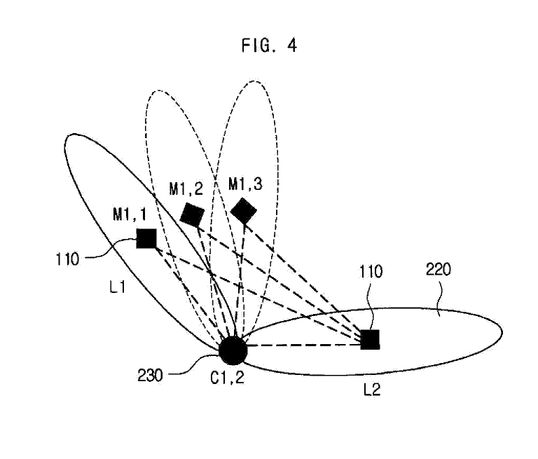

FIG. 4 illustrates an example of inducing position and velocity of a joint according to another embodiment of the present invention.

Referring to FIG. 4, it illustrates induction of position and velocity of a joint which is adjacent to body parts. It is assumed that links of the joint adjacent to body parts are assigned as L₁, L₂. The joint links(L₁, L₂) include bio-sensors 110 having different position values. The controller 130 estimates position(C₁, C₂) of the joint positioned between the adjacent body parts by using the Procrustes method.

It is assume that each of the joint links(L₁, L₂) includes one bio-sensor 110. The controller 130 determines positions of the bio-sensors 110 which are attached to the body part by continuous frames(M_{1,1} to M_{1,3}) while the body part corresponding to the joint links(L₁, L₂) moves. The controller 130 estimates position(C₁, C₂) of the joint based on the position information of the bio-sensors 110 determined by frames.

When the controller 130 obtains position(C₁, C₂) of the joint of all continuous frames(M_{1,1} to M_{1,3}), it can calculate velocity of the joint with position(C₁, C₂) of the joint for a given time.

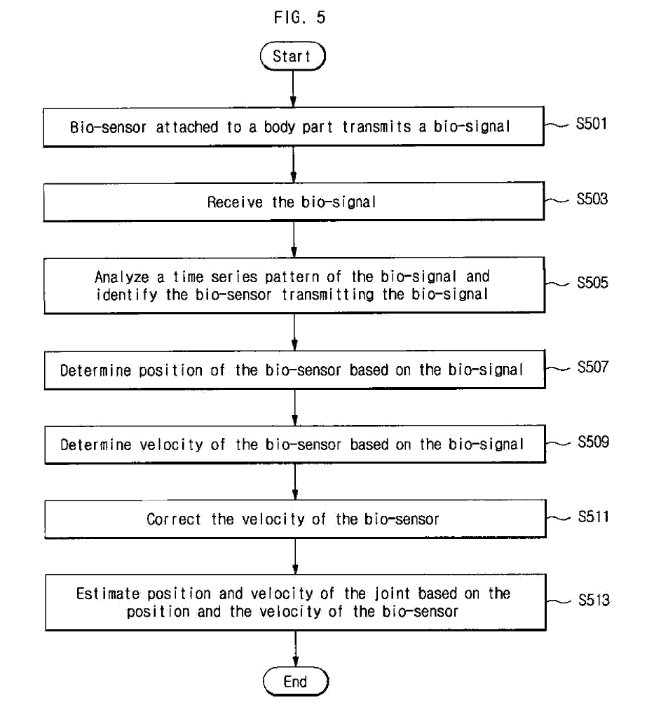

FIG. 5 is a flowchart illustrating a method for analyzing human motion according to an embodiment of the present invention.

Referring to FIG. 5, the bio-sensor 110 attached to a body part transmits a bio-signal of the body part.

In S503, the receiver 120 receives the bio-signal.

In S505, the controller 130 analyzes time series patterns of amplitudes of the bio-signals and identifies the bio-sensors 110 transmitting the bio-signal based on the analyzed result.

In S507, the controller 130 determines position of the bio-sensor 110 based on the No-signal of the bio-sensor 110. The controller 130 determines position of the bio-sensor 110 by using the triangulation and represents by the Equation(1).

In S509, the controller 130 determines velocity of the bio-sensor 110 based on the bio-signal of the bio-sensor 110. The controller 130 calculates velocity of the bio-sensor 110 by using the Equation(2). In addition, the controller 130 calculates velocity of the bio-sensor 110 through the Equation(3) using the Doppler effect.

In S511, the controller 130 interpolates the velocity of the bio-sensor 110, calculated by using the Equation(2) and the Equation(3), by using the Equation(4).

In S513, the controller 130 estimates position and velocity of the joint adjacent to the bio-sensors 110 attached to body parts based on the position and the velocity of the bio-sensor 110. The controller 130 determines position of the joint by using the Proerustes method based on the position of the bio-sensor 110. The controller 130 calculates the position of the joint included in the continuous frames(M_{1,1} to M_{1,3}) and then calculates velocity of the joint by using changes in the position of the joint.

The spirit of the present invention has been described by way of example hereinabove, and the present invention may be variously modified, altered, and substituted by those skilled in the art to which the present invention pertains without departing from essential features of the present invention.

What is claimed is:

1. An apparatus for analyzing human motion, comprising:
a bio-sensor configured to transmit a bio-signal by being attached to a body part;
a receiver configured to receive the bio-signal; and
a controller configured to determine position and velocity of the bio-sensor based on the bio-signal and estimate position and velocity of the joint adjacent to the body part based on the position and the velocity of the bio-sensor.

2. The apparatus for analyzing human motion of claim 1, wherein the controller identifies the bio-sensor transmitting the bio-signal by analyzing a time series pattern of the bio-signal.

3. The apparatus for analyzing human motion of claim 1, wherein the controller determines position of the bio-sensor by a triangulation.

4. The apparatus for analyzing human motion of claim 1, wherein the controller calculates position of the bio-sensor by using Equation of Ps(t)=(Xs(t), Ys(t), Zₛ(t)), in which Ps(t)=(Xs(t), Ys(t), Zₛ(t)) is position of the bio-sensor at time t.

5. The apparatus for analyzing human motion of claim 1, wherein the controller determines velocity of the bio-sensor by the frequency shift-based Doppler effect.

6. The apparatus for analyzing human motion of claim 1, wherein the controller calculates velocity of the bio-sensor by using Equation of
$${V}_{s}^{1} = ( \frac{{X}_{s} ( t + δ t ) - {X}_{s} ( t )}{δ t} , \frac{{Y}_{s} ( t + δ t ) - {Y}_{s} ( t )}{δ t} , \frac{{Z}_{s} ( t + δ t ) - {Z}_{s} ( t )}{δ t} ) ,$$
in which Vₛ¹ is the velocity vector of the bio-sensor at time t.

7. The apparatus for analyzing human motion of claim 1, wherein the controller calculates velocity of the bio-sensor by using Equation of
$${V}_{s}^{2} = {\sum }_{i = 1}^{3} \frac{Δ {f}_{i}}{{f}_{i}} \frac{{Q}_{i} - {P}_{s}}{‖ {Q}_{i} - {P}_{s} ‖}$$
$$‖ {Q}_{i} - {P}_{s} ‖ = {\sum }_{i = 1}^{3} \sqrt{{( {X}_{i} - {X}_{s} )}^{2} + {( {Y}_{i} - {Y}_{s} )}^{2} + {( {Z}_{i} - {Z}_{s} )}^{2}} ,$$
in which Vₛ² is the velocity vector of the bio-sensor at time t, fi is frequency of the bio-signal, Qi−Ps is a displacement vector of the bio-sensor which is a linear independent.

8. The apparatus for analyzing human motion of claim 1, wherein the controller corrects velocity of the bio-sensor by using the Equation of Vₛ=aVₛ¹+(1−a)Vₛ² (0≦a≦1), in which Vₛ is a velocity vector of the bio-sensor and a is a predetermined value by a user.

9. The apparatus for analyzing human motion of claim 1, wherein the controller estimates velocity of the joint based on the position of the joint.

10. The apparatus for analyzing human motion of claim 1, wherein the controller estimates position of the joint based on the position of the bio-sensor included in frames measured continuously in time intervals.

11. A method for analyzing human motion, comprising:
receiving a bio-signal from a bio-sensor attached to a body part;
determining position of the bio-sensor based on the bio-signal;
determining velocity of the bio-sensor based on the bio-signal; and
estimating position and velocity of the joint based on the position and the velocity of the bio-sensor.

12. The method for analyzing human motion of claim 11, further comprising identifying the bio-sensor transmitting the bio-signal by analyzing a time series pattern of the bio-signal.
